(19) United States
(12) Patent Application Publication
(10) Pub. No.: US 2022/0361507 A1
(43) Pub. Date: Nov. 17, 2022

(54) BIOCIDE AND BIOPESTICIDE COMPOSITIONS AND METHODS OF CONTROLLING ARTHROPODS

(71) Applicants: Bruno JACTEL, Brookhaven, GA (US); Ronald HATTON, JR., Raleigh, NC (US); Scot HUBER, Raleigh, NC (US); PERTINENT SANTÉ ANIMALE, SAS, Haut Mauco (FR)

(72) Inventors: Bruno JACTEL, Brookhaven, GA (US); Ronald HATTON, Jr., Raleigh, NC (US); Scott HUBER, Raleigh, NC (US)

(73) Assignee: PERTINENT SANTÉ ANIMALE, SAS, Haut Mauco (FR)

(21) Appl. No.: 17/775,854

(22) PCT Filed: Nov. 11, 2020
(86) PCT No.: PCT/US2020/059999

§ 371 Date: May 10, 2022

Related U.S. Application Data
(60) Provisional application No. 62/933,867, filed on Nov. 11, 2019.

(51) Int. Cl. A01N 65/28; A01N 31/02; A01N 65/22; A01N 31/08; A01P 7/04; A01P 7/02; A61K 31/045; A61K 36/61; A61K 36/53; A61P 33/10; A61P 33/02

(52) U.S. Cl. CPC A01N 65/28; A01N 31/02; A01N 65/22; A01N 31/08; A01P 7/04; A01P 7/02; A61K 31/045; A61K 36/61; A61K 36/53; A61P 33/10; A61P 33/02

(57) ABSTRACT

Biocide and biopesticide compositions are provided. The compositions include mixtures of essential oils and carriers, the combined effect of which provides repelling and/or killing effects against arthropod pests and internal parasites. Methods of controlling and treating for arthropod pests and internal parasites using the disclosed compositions are also provided.

### CROSS-REFERENCE TO RELATED APPLICATIONS

This application claims benefit of U.S. Provisional Patent Application Ser. No. 62/933,867, filed Nov. 11, 2019, herein incorporated by reference in its entirety.

### TECHNICAL FIELD

The presently disclosed subject matter relates to biocide and biopesticide compositions and methods of controlling arthropods and other invertebrates.

### BACKGROUND

It is estimated that by 2050 the world's population will reach 9.1 billion, 34 percent higher than today. In order to feed this larger, more urban, and richer population, food production must increase by 70 percent. Annual cereal production will need to rise to about 3 billion tons from 2.1 billion today and annual meat production will need to rise by over 200 million tons to reach 470 million tons. (1) But the necessary increased in production can be jeopardized by the detrimental impact of pests and parasites. Globally, in total of approximately 9000 species of insects and mites, 50000 species of plant pathogens, and 8000 species of weeds injure crops, of which insect pests caused an estimated 14% of loss. (2)

Therefore, pesticides are used widely in agriculture to improve yields and animal protein production. Their role is to control pests: herbicide, insecticides, nematicide, molluscicide, piscicide, avicide, rodenticide, bactericide, insect repellent, animal repellent, antimicrobial, antiprotozoal, fungicide and parasiticides for the control of internal and external parasites.

In addition, parasiticides are widely used to control Soil Transmitted Helminths in animals and humans. Soil-transmitted helminth infections are among the most common infections worldwide and affect the poorest and most deprived communities. They are transmitted by eggs present in human feces which in turn contaminate soil in areas where sanitation is poor. According to WHO, more than 1.5 billion people, or 24% of the world's population, are infected with son-transmitted helminth infections worldwide. Infections are widely distributed in tropical and subtropical areas, with the greatest numbers occurring in sub-Saharan Africa, the Americas, China, and East Asia. Over 267 million preschool-age children and over 568 million school-age children live in areas where these parasites are intensively transmitted and are in need of treatment and preventive interventions. (3)

Besides use in agriculture and human and animal health, pesticides and parasiticides are useful in professional pest control, in home and garden uses, in aquaculture, and in public health.

Chemical pesticides and parasiticides have a high toxicity profile and numerous side effects. Many of the pesticides have been associated with health and environmental issues. The numerous negative health effects that have been associated with chemical pesticides include dermatological, gastrointestinal, neurological, carcinogenic, respiratory, reproductive, and endocrine effects. Furthermore, high occupational, accidental, or intentional exposure to pesticides can result in hospitalization and death. (4) Residues of pesticides can be found in a great variety of everyday foods and beverages. Pesticides residues have also been detected in human breast milk samples, and there are concerns about prenatal exposure and health effects in children.

In addition, just as the effectiveness of antibiotics in the control of human disease is under threat due to the evolution of resistant strains of bacteria, the control of agricultural pests and crop diseases is threatened by the evolution of pesticide resistance, affecting insecticides, herbicides, parasiticides and fungicides. (5)

Therefore, because pesticides have a high toxicity profile and are less efficacious due to increased resistance, there is a need to find alternatives. Once such alternative can in some aspects be referred to as a new class of less toxic pesticides called biocides or biopesticides. What is needed are new biocides, biopesticides or other less toxic pesticides capable of repelling certain animals and insects, and/or killing certain insects and internal parasites. Such pesticides are needed that do not present any known risks to humans or the environment. Such unmet needs are addressed by the instant disclosure.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments provided herein are biocide or biopesticide compositions comprising a blend of one or more essential oils and one or more carriers, the compositions having a repelling and/or killing effect against one or more arthropod pests or internal parasites. Such blends of one or more essential oils are selected from, as examples but not limited to, geraniol, menthol, cornmint oil, peppermint oil, thyme oil, lemongrass oil, phenethylpropionate, rosemary oil, clove oil, eugenol, oregano oil, carvacrol, thymol, citronella oil, citronella, citranellol, cedarwood oil, linalool, carvacrol, para-cymene, alpha-pinene, and/or combinations thereof. Notably, the components of the compositions can have a synergistic effect.

In some aspects, the compositions and formulations can be effective in controlling internal parasites and/or helminths in humans and/or animals. Particularly, in some aspects, the compositions can be effective in controlling helminths in crops.

In some embodiments, provided herein are methods of repelling and/or killing arthropod pests. Such methods can include providing a biocide or biopesticide composition as disclosed herein, and administering the biocide or biopesticide composition to a subject or location in need of treatment, The biocide or biopesticide can be administered through a feed or foodstuff consumed by the subject, optionally a human or animal.

Provided are therapeutic compositions for treating an infection or infestation of invertebrate pests, the composition comprising any biocide or biopesticide composition disclosed herein. Provided in some aspects are methods of treating an infection or infestation of invertebrate pests in a subject, the methods comprising administering such a therapeutic composition to a subject in need, optionally wherein the subject is a human or animal subject.

In some aspects, provided are agricultural pesticide compositions comprising a biocide or biopesticide composition as disclosed herein. Correspondingly, methods of treating an infestation of invertebrate pests in an agricultural crop are provided. Such methods can include administering the pesticide composition to an agricultural crop in need, optionally wherein the agricultural crop is a plant or stored grain.

Finally, in some embodiments provided herein are animal feed supplements, the feed supplement comprising a biocide or biopesticide composition disclosed herein. Methods of treating an infestation of invertebrate pests in a domestic animal are provided, where such an animal feed supplement is fed to a livestock or domestic animal in need of treatment. Feeding the animal feed supplement to the domestic animal can treat an infestation of an invertebrate pest in the animal, increase feed efficiency.

Accordingly, these and other objects are achieved in whole or in part by the presently disclosed subject matter. Further, objects of the presently disclosed subject matter having been stated above, other objects and advantages of the presently disclosed subject matter will become apparent to those skilled in the art after a study of the following description and Examples.

### DETAILED DESCRIPTION

The presently disclosed subject matter now will be described more fully hereinafter, in which some, but not all embodiments of the presently disclosed subject matter are described. Indeed, the disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel_characteristic(s) of the claimed subject matter.

With respect to the terms “comprising”, “consisting of” and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “arthropods” refers to a Phylum that encompasses insects and acaridae (mites, ticks) as well as spiders, etc. Thus, as used herein, arthropods and insects, used sometimes interchangeably, can refer to any arthropod including, but not limited to, to mites, spiders, and other arachnids, internal parasites, helminths in adult, immature, larvae and egg forms, and like invertebrates, Also, for purposes of this application, the term “insect control” shall refer to having a repellant effect, a pesticidal effect, or both for any arthropod. Moreover, in some embodiments, “insect control” can refer to controlling/repelling arthropods (external parasites) and/or internal parasites (helminths and protozoa).

As used herein, the terms “repel”, “inhibit”, “suppress”, “repress”, “kill”, “control”, “efficacy”, and grammatical variants thereof are used interchangeably and refer to an activity whereby arthropod, invertebrate or insect activity and/or viability is reduced below that observed in the absence of a composition or formulation of the presently disclosed subject matter. In some embodiments, arthropod, invertebrate or insect control that results in repelling or inhibiting activity results in a decrease, e.g. a measurable decrease, in the presence and/or survivability of an arthropod, invertebrate or insect in an environment or on/in a subject, particularly as compared to an untreated environment or subject.

### II. Subjects

In some embodiments a subject treated or administered a disclosed composition is desirably a human subject, although it is to be understood that the principles of the disclosed subject matter indicate that the compositions and methods are effective with respect to invertebrate and to all vertebrate species, including mammals, such as domestic animals, livestock animals, companion animals, rodents, etc., which are intended to be included in the term “subject”. Moreover, a mammal is understood to include any mammalian species in which treatment is desirable, particularly agricultural and domestic mammalian species.

The disclosed methods and treatments are particularly useful in the treatment of warm-blooded vertebrates. Thus, the presently disclosed subject matter concerns mammals and birds.

More particularly, provided herein is the treatment of mammals such as humans, as well as those mammals of importance due to being laboratory animals for modeling human diseases and conditions (such as mice, rats and pigs), endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Also provided is the treatment of fish, including fish in commercial aquaculture operations, as well as ornamental fish in home, professional, and commercial aquaria.

### III. Discussion

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Disclosed herein is a series of formulations made of combinations of essential oils with demonstrated activities in multiple species of arthropods, e.g., insects and external parasites, as well as internal parasites, e.g. helminths and protozoa, with potential applications in agriculture, human health and animal health. In some embodiments, the essential oil blends can have a synergistic effect on controlling arthropods and internal parasites.

In some embodiments, provided herein are biocide, biopesticide, or parasiticide compositions, the compositions comprising a blend of one or more essential oils and one or more carriers, the compositions having a repelling and/or killing effect against one or more arthropods, including for example insect pests and internal parasites. In some aspects, such biocide or biopesticide compositions can comprise one or more of thymol, geraniol, linalool, carvacrol, para-cymene, alpha-pinene, menthol, eugenol, carvacrol, citronella, citronellol, cinnamaldehyde, cinnamol, comm int oil, peppermint oil, thyme oil, lemongrass oil, rosemary oil, oregano oil, clove oil, citronella oil, cinnamon oil, and/or combinations thereof. In some embodiments, such compositions can further comprise one or more carriers, including for example, but not limited to, white mineral oil, triethyl citrate, isopropyl myristate, sodium bicarbonate, calcium silicate, calcium carbonate, water, isopropanol, ethanol, soap, fatty acid salts or esters, sodium lauryl sulfate. In some embodiments, the components of the compositions, including but not limited to essential oils and carriers, can have a synergistic effect in controlling arthropods, external parasites, and other invertebrates. Additionally, such compositions can have a synergistic effective in controlling arthropods, nematodes, helminths, external parasites, internal parasites and/or invertebrate, in humans, animals and/or crops, for professional pest control, for use in homes and gardens, or for use in aquaculture.

In some embodiments, and as described further herein in the Examples and formulations (Tables 1-3), a biocide or biopesticide composition can, for example, comprise geraniol, triethyl citrate and white mineral oil. By way of example only, such compositions can comprise about 3-6% geraniol (e.g. 3%, 4%, 5%, 6% geraniol), 1-4% triethyl citrate (e.g. 1%, 2%, 3%, 4% triethyl citrate) and 90-96% mineral oil, e.g, white mineral oil (e.g. 90%, 91%, 92%, 93%, 94%, 95%, 96% mineral oil). Additionally, by way of example only, such biocide or biopesticide compositions can comprise geraniol, isopropyl myristate and white mineral oil, for example about 3-6% geraniol (e.g. 3%, 4%, 5%, 6% geraniol), 1-4% isopropyl myristate (e.g. 1%, 2%, 3%, 4% isopropyl myristate) and 90-96% mineral oil/white mineral oil (e.g. 90%, 91%, 92%, 93%, 94%, 95%, 96% mineral on). A complete listing of example formulations and compositions are provided below in Tables 1, 2 and 3. Where ranges of concentrations are provided herein and in the tables, e.g. 3-8%, all intermediate concentrations, e.g. 3%, 4%, 5%, 6%, 7% and 8%, within the stated range are included and provided herein.

TABLE 1
Example Compositions

| Ingredient | 1 | 2 | 3 | 4 | 5 | 6 | 7 | 8 | 9 | 10 |
|---|---|---|---|---|---|---|---|---|---|---|
| Geraniol | | 0.5-3% | 0.5-3% | 0.5-3% | 13-8% | 13-8% | 0.5-3% | 0.5-3% | 0.5- | 0.5- |
| | | | | | | | | | 3% | 3% |
| Menthol | | | | | | | 0.5-3% | | | |
| Cornmint | | | | | | | | 0.5-3% | 0.5- | |
| oil | | | | | | | | | 3% | |
| Peppermint | | | | | | | | | | 0.5- |
| oil | | | | | | | | | | 3% |
| Thyme oil | | | | | | | | | | |
| Lemongrass | | | | | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 8-14% | 3-8% | 3-8% | 0.5-3% | 63-8% | 8-14% | 3-8% | 3-8% | 3-8% | 3-8% |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | |
| myristate | | | | | | | | | | |
| Butyl lactate | | | | | | | | | | |
| Triethyl | | | | | | | | | | |
| citrate | | | | | | | | | | |
| Ethyl lactate | 0.5-3% | | | | | | | | | |
| Glycerin | 0.5-3% | 0.5-3% | 0.5-3% | 0.5-3% | | | 0.5-3% | 0.5-3% | | |
| Isopropanol | 3-8% | 3-8% | 3-8% | 3-8% | 14-20% | | 3-8% | 3-8% | 3-8% | 3-8% |
| Water | 50-80% | 80-90% | 80-90% | 90-95% | | 14-20% | 80-90% | 80-90% | 80- | 80- |
| | | | | | | | | | 90% | 90% |
| Mineral oil | | | | | | 50-80% | | | | |
| Ingredient | 11 | 12 | 13 | 14 | 15 | 16 | 17 | 18 | 19 | 20 |
| Geraniol | 0.5-3% | 0.5-3% | 0.5-3% | 0.5-3% | 0.5-3% | 0.5-3% | 0.5-3% | 0.5-3% | 0.5- | 0.5- |
| | | | | | | | | | 3% | 3% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | 0.5-3% | 0.5-3% | 0.5-3% | | | | | | | |
| Lemongrass | | | | 0.01-0.5% | | | | | | |
| oil | | | | | | | | | | |
| Phenethyl- | | | | | 0.01-0.5% | | | | | |
| propionate | | | | | | | | | | |
| Rosemary | | | | | | 0.01- | | | | |
| oil | | | | | | 0.5% | | | | |
| Clove oil | | | | | | | 0.01-0.5% | | | |
| Eugenol | | | | | | | | 0.01-0.5% | | |
| Oregano | | | | | | | | | 0.01- | |
| oil | | | | | | | | | 0.5% | |
| Carvacrol | | | | | | | | | | 0.01- |
| | | | | | | | | | | 0.5% |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 3-8% | 3-8% | 0.5-3% | 0.5-3% | 0.5-3% | 0.5-3% | 0.5-3% | 0.5-3% | 0.5- | 0.5- |
| | | | | | | | | | 3% | 3% |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | |
| myristate | | | | | | | | | | |
| Butyl lactate | | | | | | | | | | |
| Triethyl | | | | | | | | | | |
| citrate | | | | | | | | | | |
| Ethyl lactate | | | | | | | | | | |
| Glycerin | | | | | | | | | | |
| Isopropanol | 3-8% | 3-8% | 3-8% | 3-8% | 3-8% | 3-8% | 3-8% | 3-8% | 3-8% | 3-8% |
| Water | 80-90% | 80-90% | 80-90% | 80-90% | 80-90% | 80-90% | 80-90% | 80-90% | 80- | 80- |
| | | | | | | | | | 90% | 90% |
| Mineral oil | | | | | | | | | | |
| Ingredient | 21 | 22 | 23 | 24 | 25 | 26 | 27 | 28 | 29 | 30 |
| Geraniol | 0.5-3% | 0.5-3% | 0.5-3% | 0.5-3% | 0.5-3% | 0.5-3% | 0.5-3% | 0.5-3% | 0.5- | 0.5- |
| | | | | | | | | | 3% | 3% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | | | | | | | | |
| Lemongrass | | | | | | 0.5-3% | | | | |
| oil | | | | | | | | | | |
| Phenethyl- | | | | | | | 0.5-3% | | | |
| propionate | | | | | | | | | | |
| Rosemary | | | | | | | | 0.5-3% | | |
| oil | | | | | | | | | | |
| Clove oil | | | | | | | | | 0.5- | |
| | | | | | | | | | 3% | |
| Eugenol | | | | | | | | | | 0.5- |
| | | | | | | | | | | 3% |
| Oregano | | | | | | | | | | |
| oil | | | | | | | | | | |
| Carvacrol | | | | | | | | | | |
| Thymol | 0.01-0.5% | | | | | | | | | |
| Citronella | | 0.01-0.5% | | | | | | | | |
| oil | | | | | | | | | | |
| Citronella | | | 0.01-0.5% | | | | | | | |
| Citranellol | | | | 0.01-0.5% | | | | | | |
| Cedarwood | | | | | 0.01-0.5% | | | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 0.5-3% | 0.5-3% | 0.5-3% | 0.5-3% | 0.5-3% | 0.5-3% | 0.5-3% | 0.5-3% | 0.5- | 0.5- |
| | | | | | | | | | 3% | 3% |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | |
| myristate | | | | | | | | | | |
| Butyl lactate | | | | | | | | | | |
| Triethyl | | | | | | | | | | |
| citrate | | | | | | | | | | |
| Ethyl lactate | | | | | | | | | | |
| Glycerin | | | | | | | | | | |
| Isopropanol | 3-8% | 3-8% | 3-8% | 3-8% | 3-8% | 3-8% | 3-8% | 3-8% | 3-8% | 3-8% |
| Water | 80-90% | 80-90% | 80-90% | 80-90% | 80-90% | 80-90% | 80-90% | 80-90% | 80- | 80- |
| | | | | | | | | | 90% | 90% |
| Mineral oil | | | | | | | | | | |
| Ingredient | 31 | 32 | 33 | 34 | 35 | 36 | 37 | 38 | 39 | 40 |
| Geraniol | 0.5-3% | 0.5-3% | 0.5-3% | 0.5-3% | 0.5-3% | 0.5-3% | 0.5-3% | 0.5-3% | 0.5- | 0.5- |
| | | | | | | | | | 3% | 3% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | | | | | | | | |
| Lemongrass | | | | | | | | 3-8% | | |
| oil | | | | | | | | | | |
| Phenethyl- | | | | | | | | | 3-8% | |
| propionate | | | | | | | | | | |
| Rosemary | | | | | | | | | | 3-8% |
| oil | | | | | | | | | | |
| Clove oil | | | | | | | | | | |
| Eugenol | | | | | | | | | | |
| Oregano | 0.5-3% | | | | | | | | | |
| oil | | | | | | | | | | |
| Carvacrol | | 0.5-3% | | | | | | | | |
| Thymol | | | 0.5-3% | | | | | | | |
| Citronella | | | | 0.5-3% | | | | | | |
| oil | | | | | | | | | | |
| Citronella | | | | | 0.5-3% | | | | | |
| Citranellol | | | | | | 0.5-3% | | | | |
| Cedarwood | | | | | | | 0.5-3% | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 0.5-3% | 0.5-3% | 0.5-3% | 0.5-3% | 0.5-3% | 0.5-3% | 0.5-3% | 0.5-3% | 0.5- | 0.5- |
| | | | | | | | | | 3% | 3% |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | |
| myristate | | | | | | | | | | |
| Butyl lactate | | | | | | | | | | |
| Triethyl | | | | | | | | | | |
| citrate | | | | | | | | | | |
| Ethyl lactate | | | | | | | | | | |
| Glycerin | | | | | | | | | | |
| Isopropanol | 3-8% | 3-8% | 3-8% | 3-8% | 3-8% | 3-8% | 3-8% | 3-8% | 3-8% | 3-8% |
| Water | 80-90% | 80-90% | 80-90% | 80-90% | 80-90% | 80-90% | 80-90% | 80-90% | 80- | 80- |
| | | | | | | | | | 90% | 90% |
| Mineral oil | | | | | | | | | | |
| Ingredient | 41 | 42 | 43 | 44 | 45 | 46 | 47 | 48 | 49 | 50 |
| Geraniol | 0.5-3% | 0.5-3% | 0.5-3% | 0.5-3% | 0.5-3% | 0.5-3% | 0.5-3% | 0.5-3% | 0.5- | 8- |
| | | | | | | | | | 3% | 14% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | | | | | | | | |
| Lemongrass | | | | | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | 3-8% | | | | | | | | | |
| Eugenol | | 3-8% | | | | | | | | |
| Oregano | | | 3-8% | | | | | | | |
| oil | | | | | | | | | | |
| Carvacrol | | | | 3-8% | | | | | | |
| Thymol | | | | | 3-8% | | | | | |
| Citronella | | | | | | 3-8% | | | | |
| oil | | | | | | | | | | |
| Citronella | | | | | | | 3-8% | | | |
| Citranellol | | | | | | | | 3-8% | | |
| Cedarwood | | | | | | | | | 3-8% | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 0.5-3% | 0.5-3% | 0.5-3% | 0.5-3% | 0.5-3% | 0.5-3% | 0.5-3% | 0.5-3% | 0.5- | 8- |
| | | | | | | | | | 3% | 14% |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | 20- |
| myristate | | | | | | | | | | 50% |
| Butyl lactate | | | | | | | | | | |
| Triethyl | | | | | | | | | | |
| citrate | | | | | | | | | | |
| Ethyl lactate | | | | | | | | | | |
| Glycerin | | | | | | | | | | |
| Isopropanol | 3-8% | 3-8% | 3-8% | 3-8% | 3-8% | 3-8% | 3-8% | 3-8% | 3-8% | |
| Water | 80-90% | 80-90% | 80-90% | 80-90% | 80-90% | 80-90% | 80-90% | 80-90% | 80- | 20- |
| | | | | | | | | | 90% | 50% |
| Mineral oil | | | | | | | | | | |
| Ingredient | 51 | 52 | 53 | 54 | 55 | 56 | 57 | 58 | 59 | 60 |
| Geraniol | 20-40% | 0.5-3% | 0.5-3% | 0.5-3% | 3-8% | 0.5-3% | 0.5-3% | 0.5-3% | 20- | 53- |
| | | | | | | | | | 40% | 8% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | 0.5-3% | 0.5-3% | | 0.5-3% | 0.5-3% | 0.5-3% | | |
| Lemongrass | | | | | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | | | | | 8- | 0.01- |
| lauryl | | | | | | | | | 14% | 0.5% |
| sulfate | | | | | | | | | | |
| Lecithin | | | | | | | | | | 3-8% |
| Polyglyceryl | | | | | | | | | | |
| oleate | | | | | | | | | | |
| Soap | 8-14% | 3-8% | 3-8% | 3-8% | 3-8% | 3-8% | 3-8% | 3-8% | | |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | |
| myristate | | | | | | | | | | |
| Butyl lactate | | | | | | | | | | 20- |
| | | | | | | | | | | 50% |
| Triethyl | | | | | | | | | | |
| citrate | | | | | | | | | | |
| Ethyl lactate | | | | | | | | | | |
| Glycerin | | | | | | | | | | |
| Isopropanol | | 3-8% | 3-8% | 3-8% | 8-14% | 3-8% | 3-8% | 8-14% | 8- | |
| | | | | | | | | | 14% | |
| Water | 20-50% | 80-90% | 80-90% | 80-90% | 80-90% | 80-90% | 80-90% | 80-90% | 20- | |
| | | | | | | | | | 50% | |
| Mineral oil | 8-14% | | | | | | | | | |
| Ingredient | 61 | 62 | 63 | 64 | 65 | 66 | 67 | 68 | 69 | 70 |
| Geraniol | 0.5-3% | 0.5-3% | 0.5-3% | 3-8% | 0.5-3% | 0.5-3% | 0.5-3% | 0.5-3% | 0.5- | 0.5- |
| | | | | | | | | | 3% | 3% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | 0.5-3% | 0.5-3% | 0.5-3% | | 0.5-3% | 0.5-3% | 0.5-3% | 0.5-3% | | |
| Lemongrass | | | | | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 3-8% | 3-8% | 3-8% | 3-8% | 3-8% | 3-8% | 3-8% | 3-8% | 0.5- | 3-8% |
| | | | | | | | | | 3% | |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | | |
| myristate | | | | | | | | | | |
| Butyl lactate | | | | | | | 0.5-3% | 0.5-3% | | |
| Triethyl | | | | | | | | | | |
| citrate | | | | | | | | | | |
| Ethyl lactate | | | | | 0.5-3% | 0.5-3% | | | | |
| Glycerin | 0.5-3% | 0.5-3% | | | | | | | | |
| Isopropanol | 3-8% | 3-8% | 8-14% | 8-14% | 3-8% | 3-8% | 3-8% | 3-8% | 3-8% | 3-8% |
| Water | 80-90% | 80-90% | 50-80% | 80-90% | 50-80% | 80-90% | 50-80% | 80-90% | 90- | 80- |
| | | | | | | | | | 95% | 90% |
| Mineral oil | | | | | | | | | | |
| Ingredient | 71 | 72 | 73 | 74 | 75 | 76 | 77 | 78 | 79 | 80 |
| Geraniol | 0.5-3% | 0.5-3% | 0.5-3% | 0.5-3% | 0.5-3% | 0.5-3% | 0.5-3% | 3-8% | 3-8% | 0.5- |
| | | | | | | | | | | 3% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | 0.5-3% | 0.5-3% | | | | | | | |
| Lemongrass | | | | | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | 3-8% | 3-8% | 3-8% | 0.5-3% | 0.5-3% | 3-8% | 0.5-3% | | | |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | | | | | | | | | 0.5- | 0.5- |
| myristate | | | | | | | | | 3% | 3% |
| Butyl lactate | | | | | | | | | | |
| Triethyl | | | | | | | | 0.5-3% | | |
| citrate | | | | | | | | | | |
| Ethyl lactate | | | | | | | | | | |
| Glycerin | | | | | | | | | | |
| Isopropanol | 3-8% | 3-8% | 3-8% | 3-8% | 3-8% | 3-8% | 3-8% | | | |
| Water | 80-90% | 80-90% | 80-90% | 90-95% | 90-95% | 80-90% | 90-95% | | | |
| Mineral oil | | | | | | | | 90-95% | 90- | 90- |
| | | | | | | | | | 95% | 95% |
| Ingredient | 81 | 82 | 83 | 84 | 85 | 86 | 87 | 88 | 89 | 90 |
| Geraniol | 0.5-3% | 0.01-0.5% | 3-8% | 0.5-3% | 0.5-3% | 0.5-3% | | 0.5-3% | 0.01- | 0.01- |
| | | | | | | | | | 0.5% | 0.5% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | | | | | | | | |
| Lemongrass | | | | | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | | | | | | |
| lauryl | | | | | | | | | | |
| sulfate | | | | | | | | | | |
| Lecithin | | | | | | | | | | |
| Polyglyceryl | | | | | | | | | | |
| oleate | | | | | | | | | | |
| Soap | | | 8-14% | 8-14% | 8-14% | 8-14% | | | | |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | 0.5-3% | 0.5-3% | | | | | | | 0.5- | 0.5- |
| myristate | | | | | | | | | 3% | 3% |
| Butyl lactate | | | | | | | | | | |
| Triethyl | | | | | | | | | | |
| citrate | | | | | | | | | | |
| Ethyl lactate | | | 3-8% | 3-8% | 3-8% | 3-8% | | | | |
| Glycerin | | | 0.5-3% | 0.5-3% | 0.5-3% | 0.5-3% | | | | |
| Isopropanol | | | 0.5-3% | 0.5-3% | 0.5-3% | 0.5-3% | 3-8% | 3-8% | | |
| Water | | | | | | | | | | |
| Mineral oil | 95- | 95- | 50-80% | 50-80% | 50-80% | 50-80% | 90-95% | 90-95% | 95- | 95- |
| | 99.99% | 99.99% | | | | | | | 99.99% | 99.99% |
| Ingredient | 91 | 92 | 93 | 94 | 95 | 96 | 97 | 98 | 99 | 100 |
| Geraniol | 8-14% | 0.5-3% | 0.5-3% | | 3-8% | 3-8% | 3-8% | 3-8% | 3-8% | 3-8% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | | | | | | | | |
| Lemongrass | | | | | | | | | | |
| oil | | | | | | | | | | |
| Phenethyl- | | | | | | | | 3-8% | 3-8% | 3-8% |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | | | | | 3-8% | | | | | |
| lauryl | | | | | | | | | | |
| sulfate | | | | | | | | | | |
| Lecithin | | | | | | 0.5-3% | 0.5-3% | | | |
| Polyglyceryl | | | | | 0.5-3% | 0.5-3% | 0.5-3% | | | |
| oleate | | | | | | | | | | |
| Soap | | | 3-8% | 3-8% | | | | 8-14% | | |
| Sodium | | | | | | | | | | |
| stearate | | | | | | | | | | |
| Potassium | | | | | | | | | | |
| oleate | | | | | | | | | | |
| Potassium | | | | | | | | | | |
| laurate | | | | | | | | | | |
| Isopropyl | 0.5-3% | 0.5-3% | | | | | | | | |
| myristate | | | | | | | | | | |
| Butyl lactate | | | | | | | | | | |
| Triethyl | | | | | 3-8% | | | | | 0.5- |
| citrate | | | | | | | | | | 3% |
| Ethyl lactate | | | | | | | | | | |
| Glycerin | | | | | 3-8% | | 0.5-3% | 0.5-3% | | |
| Isopropanol | | | | | | | | 8-14% | | |
| Water | | | | | 80-90% | 90-95% | 90-95% | 50-80% | | |
| Mineral oil | 80-90% | 90-95% | 90-95% | 90-95% | | | | | 90- | 90- |
| | | | | | | | | | 95% | 95% |
| Ingredient | 101 | 102 | 103 | 104 | 105 | 106 | 107 | 108 | 109 | 110 |
| Geraniol | 3-8% | 3-8% | 3-8% | 0.5-3% | 3-8% | 3-8% | 0.5-3% | 0.5-3% | 0.5- | 0.01- |
| | | | | | | | | | 3% | 0.5% |
| Menthol | | | | | | | | | | |
| Cornmint | | | | | | | | | | |
| oil | | | | | | | | | | |
| Peppermint | | | | | | | | | | |
| oil | | | | | | | | | | |
| Thyme oil | | | | | | | | | | |
| Lemongrass | | | | 0.5-3% | | | | | | |
| oil | | | | | | | | | | |
| Phenethyl- | | | | | | | | | | |
| propionate | | | | | | | | | | |
| Rosemary | | | | | | | | | | |
| oil | | | | | | | | | | |
| Clove oil | | | | | | | | | | |
| Eugenol | | | | | | | | | | |
| Oregano | | | | | | | | | | |
| oil | | | | | | | | | | |
| Carvacrol | | | | | | | | | | |
| Thymol | | | | | | | | | | |
| Citronella | | | | | | | | | | |
| oil | | | | | | | | | | |
| Citronella | | | | | | | | | | |
| Citranellol | | | | | | | | | | |
| Cedarwood | | | | | | | | | | |
| oil | | | | | | | | | | |
| Sodium | 0.5-3% | | | | | | | | | |
| lauryl | | | | | | | | | | |
| sulfate | | | | | | | | | | |
| Lecithin | | | | | | | | | | |
| Polyglyceryl | 0.5-3% | | | | | | | | | |
| oleate | | | | | | | | | | |
| Soap | | | | | | | | | | 0.5- |
| Sodium | | 10.5-3% | | | | | | | | 3% |
| stearate | | | | | | | | | | |
| Potassium | | | 3-8% | | | | | | | |
| oleate | | | | | | | | | | |
| Potassium | | | | | 8-14% | 8-14% | | | | |
| laurate | | | | | | | | | | |
| Isopropyl | | | | | | | 0.5-3% | 0.5-3% | 0.5- | |
| myristate | | | | | | | | | 3% | |
| Butyl lactate | | | | | | | | | | |
| Triethyl | | | | 0.5-3% | | | | | | |
| citrate | | | | | | | | | | |
| Ethyl lactate | 3-8% | | 0.5-3% | | | 0.5-3% | | | | |
| Glycerin | | | 0.5-3% | | 0.5-3% | 0.5-3% | | | | |
| Isopropanol | | 3-8% | 8-14% | | 8-14% | 8-14% | | | | 3-8% |
| Water | 80-90% | 50-80% | 50-80% | | | 50-80% | | | | 90- |
| | | | | | | | | | | 95% |
| Mineral oil | | | | 90-95% | 50-80% | | 90-95% | 95- | 95- | |
| | | | | | | | | 99.99% | 99.99% | |

| Ingredient | 111 |
|---|---|
| Geraniol | 0.01-0.5% |
| Menthol | |
| Cornmint | |
| oil | |
| Peppermint | |
| oil | |
| Thyme oil | |
| Lemongrass | |
| oil | |
| Phenethyl- | |
| propionate | |
| Rosemary | |
| oil | |
| Clove oil | |
| Eugenol | |
| Oregano | |
| oil | |
| Carvacrol | |
| Thymol | |
| Citronella | |
| oil | |
| Citronella | |
| Citranellol | |
| Cedarwood | |
| oil | |
| Sodium | |
| lauryl | |
| sulfate | |
| Lecithin | |
| Polyglyceryl | |
| oleate | |
| Soap | |
| Sodium | |
| stearate | |
| Potassium | |
| oleate | |
| Potassium | |
| laurate | |
| Isopropyl | 0.5-3% |
| myristate | |
| Butyl lactate | |
| Triethyl | |
| citrate | |
| Ethyl lactate | |
| Glycerin | |
| Isopropanol | |
| Water | |
| Mineral oil | 95-99.99% |

TABLE 2
Example compositions

| Ingredient | 112 | 113 | 114 | 115 | 116 | 117 | 118 |
|---|---|---|---|---|---|---|---|
| Geraniol | 3-8% | 3-8% | 3-8% | 3-8% | 3-8% | 3-8% | 3-8% |
| Thyme oil | 3-8% | 3-8% | 3-8% | 3-8% | 3-8% | 3-8% | 3-8% |
| Calcium silicate | 3-8% | 3-8% | 3-8% | 3-8% | 8-14% | 3-8% | 3-8% |
| Calcium carbonate | 3-8% | 8-14% | 14-20% | 20-40% | 8-14% | 3-8% | 3-8% |
| Sodium bicarbonate | 80-90% | 50-80% | 50-80% | 50-80% | 50-80% | 80-90% | 80-90% |

TABLE 3
Example compositions

| Ingredient | 119 | 120 | 121 | 122 | 123 | 124 | 125 | 126 | 127 | 128 | 129 | |
|---|---|---|---|---|---|---|---|---|---|---|---|---|
| Thymol | 100% | | | 50-80% | 50-80% | 50-80% | 50-80% | | | | 20-50% | |
| Linalool | | 100% | | 14-20% | 14-20% | 14-20% | | 14-20% | 14-20% | 14-20% | 14-20% | |
| Carvacrol | | | 100% | | | | 50-80% | 50-80% | 50-80% | 70 | 20-50% | |
| para-Cymene | | | | | 14-20% | | | | 14-20% | | | |
| alpha-Pinene | | | | | | 14-20% | | | | 14-20% | | |
| Sodium | | | | | | | | | | | | |
| caseinate | | | | | | | | | | | | |
| Hydrogenated | | | | | | | | | | | | |
| palm oil | | | | | | | | | | | | |
| Ingredient | 130 | 131 | 132 | 133 | 134 | 135 | 136 | 137 | 138 | 139 | 140 | 141 |
| Thymol | 14-20% | | 8-14% | 8-14% | 8-14% | 8-14% | 3-8% | | | | 8-14% | 8-14% |
| Linalool | | 14-20% | 0.01- | 0.5-3% | 0.5-3% | 0.5-3% | | 0.5-3% | 0.5-3% | 0.5-3% | 3-8% | 3-8% |
| | | | 0.5% | | | | | | | | | |
| Carvacrol | | | | | | 0.01- | 3-8% | 8-14% | 8-14% | 8-14% | 8-14% | 8-14% |
| | | | | | | 0.5% | | | | | | |
| para-Cymene | | | | | 0.5-3% | | | | 0.5-3% | | | |
| alpha-Pinene | | | | | | | | | | 0.5-3% | | |
| Sodium | 80-90% | 50-80% | 80-90% | 80-90% | 80-90% | 80-90% | 80-90% | 80-90% | 80-90% | 80-90% | 50-80% | |
| caseinate | | | | | | | | | | | | |
| Hydrogenated | | | | | | | | | | | | 50-80% |
| palm oil | | | | | | | | | | | | |

In some aspects, the geraniol can comprise any level of purity, including for example geraniol 98%. As a further example, a biocide or biopesticide composition can comprise geraniol 98%, thyme oil, calcium silicate, calcium carbonate, and sodium bicarbonate, optionally about 3-6% geraniol 98%, 3-6% thyme oil, 10-15% calcium silicate, 10-15% calcium carbonate and 64-77% sodium bicarbonate.

Also provided herein are methods of repelling and/or killing arthropods, including insect pests and internal parasites. Such methods can include providing a biocide or biopesticide composition as disclosed herein, and administering the biocide or biopesticide composition to a subject or location in need of treatment. In some embodiments, such biocides or biopesticides can be added to feedstuffs or food of animals and humans such that upon consumption by animals and/or humans internal parasites can be treated. For example, such methods can be used on humans, animals (including livestock and/or domesticated pets), and in any location or premises, e.g. agricultural crops, with an arthropod or insect or internal parasite infestation.

In some embodiments, provided herein are biocide and/or biopesticide compositions that are encapsulated (8) and/or configured to be encapsulated. For example, in some embodiments, biocide and/or biopesticide compositions comprising thymol, carvacrol, linalool, etc. can be encapsulated to enhance efficacy.

To elaborate, in some embodiments provided herein are biocide or biopesticide compositions comprising a blend of one or more essential oils and one or more carriers, the compositions having a repelling and/or killing effect against one or more arthropod pests or internal parasites. Such blends of one or more essential oils are selected from, as examples but not limited to, geraniol, menthol, cornmint oil, peppermint oil, thyme oil, lemongrass oil, phenethylpropionate, rosemary oil, clove oil, eugenol, oregano oil, carvacrol, thymol, citronella oil, citronella, citranellol, cedarwood oil, linalool, carvacrol, para-cymene, alpha-pinene, and/or combinations thereof. Notably, the components of the compositions can have a synergistic effect.

In some aspects, the compositions and formulations can be effective in controlling internal parasites and/or helminths in humans and/or animals. Particularly, in some aspects, the compositions can be effective in controlling helminths in crops.

As a specific example, and as discussed in the Examples and shown in Tables 1-3 above, a biocide or biopesticide composition (e.g. formulation 29) can comprise geraniol, clove oil, soap, isopropanol and water, optionally about 0.5% to about 3.0% geraniol, about 0.5% to about 3.0% clove oil, about 0.5% to about 3.0% soap, about 3.0% to about 8.0% isopropanol and about 80.0% to about 90.0% water. As another example, a biocide or biopesticide composition (e.g. formulations 69-77) can comprise geraniol, soap, isopropanol and water, optionally about 0.5% to about 3.0% geraniol, about 0.5% to about 8.0% soap, about 3.0% to about 8.0% isopropanol and about 80.0% to about 95.0% water. A further example (e.g. formulation 78) can comprise geraniol, triethyl citrate and mineral oil, optionally about 3.0% to about 8.0% geraniol, about 0.5% to about 3.0% triethyl citrate and about 90% to about 95% mineral oil (optionally mineral oil). Yet a further composition or formulation (e.g. formulation 79) can include, for example, geraniol, isopropyl myristate and mineral oil, optionally about 3.0% to about 8.0% geraniol, about 0.5% to about 3.0% isopropyl myristate and about 90% to about 95% mineral oil.

In some embodiments, a further example formulation (e.g. formulations 83-86) can comprise geraniol, soap, ethyl lactate, glycerine, isopropanol and mineral oil, optionally about 0.5% to about 8,0% geraniol, about 8.0% to about 14.0% soap, about 3.0% to about 8.0% ethyl lactate, about 0.5% to about 3.0% glycerine, about 0.5% to about 3.0% isopropanol, and about 50% to about 80% mineral oil. Another example (e.g. formulations 107-109) can comprise geraniol, isopropyl myristate and mineral oil, optionally about 3.0% to about 8.0% geraniol, about 0.5% to about 3.0% isopropyl myristate and about 90% to about 95% mineral oil. Another example (e.g. formulation 110) can comprise geraniol, soap, isopropanol and water, optionally about 0.01% to about 0.5% geraniol, about 0.5% to about 3.0% soap, about 3.0% to about 8.0% isopropanol, and about 90% to about 95% water. Another example formulation can comprise (e.g. formulation 111) geraniol, isopropyl myristate and mineral oil, optionally about 0.01% to about 0.5% geraniol, about 0.5% to about 3.0% isopropyl myristate, and about 95.0% to about 99.99% water. Other formulations and compositions not listed above are provided in Tables 1, 2 and 3, and discussed in the Examples.

In some aspects, the biocide or biopesticide compositions comprise geraniol having a purity ranging from about 5% to about 100%. In some aspects, the biocide or biopesticide compositions are in a liquid form, or in a substantially liquid state.

In some aspects, another example composition (e.g. formulations 112-118) can comprise geraniol, thyme oil, calcium silicate, calcium carbonate and sodium bicarbonate, optionally about 3.0 to about 8.0% geraniol , about 3.0% to about 8.0% thyme oil, about 3.0% to about 14% calcium silicate, about 3,0% to about 40% calcium carbonate, and about 50% to about 90% sodium bicarbonate. Such compositions can be a solid, or substantially solid form.

In some aspects, example biocide or biopesticide compositions can comprise one or both of thymol and sodium caseinate, and optionally one or more of linalool, carvacrol, para-cymene, and alpha-pinene. The thymol can be at about 50% to about 100%. Alternatively, the thymol can be at about 3% to about 50%, and sodium caseinate at about 80% to about 90%. In some aspects, the composition can be configured for the treatment of internal parasites in a subject. See, e.g. formulations 119-141 in Table 3.

In some embodiments, provided herein are methods of repelling and/or killing arthropod pests. Such methods can include providing a biocide or biopesticide composition as disclosed herein, and administering the biocide or biopesticide composition to a subject or location in need of treatment. The biocide or biopesticide can be administered through a feed or foodstuff consumed by the subject, optionally a human or animal.

Provided are therapeutic compositions for treating an infection or infestation of invertebrate pests, the composition comprising any biocide or biopesticide composition disclosed herein. Provided in some aspects are methods of treating an infection or infestation of invertebrate pests in a subject, the methods comprising administering such a therapeutic composition to a subject in need, optionally wherein the subject is a human or animal subject.

In some aspects, provided are agricultural pesticide compositions comprising a biocide or biopesticide composition as disclosed herein. Correspondingly, methods of treating an infestation of invertebrate pests in an agricultural crop are provided. Such methods can include administering the pesticide composition to an agricultural crop in need, optionally wherein the agricultural crop is a plant or stored grain.

Finally, in some embodiments provided herein are animal feed supplements, the feed supplement comprising a biocide or biopesticide composition disclosed herein. Methods of treating an infestation of invertebrate pests in a domestic animal are provided, where such an animal feed supplement is fed to a livestock or domestic animal in need of treatment. Feeding the animal feed supplement to the domestic animal can treat an infestation of an invertebrate pest in the animal, increase feed efficiency.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

In one embodiment, provided are several blends (Table 1) comprising in a synergistic combinations in various proportions. These combinations have demonstrated efficacy against crawling and flying insects.

When tested Examples 78, 79, 83, 107, 108, 109, and 111 afforded greater than about 90% mortality against beetles (A. diaperinus) when applied to the beetles as a spray.

Likewise, the testing of Examples 78, 79, 83, 84, 85, 86, 107, and 110 afforded greater than about 90% mortality against house flies (M. domestica) when applied to the flies as a spray.

### Example 2

In some embodiments, provided are several blends (Table 1) comprising a synergistic combination in various proportions and combinations. These combinations when tested demonstrated up to 100% efficacy against insects and parasites in agriculture, animal, and human health.

Test materials and compositions were applied to the floor and walls of chicken production houses, infested with litter beetles and their larvae, at a rate of about 20 L to about 40 L per 1,000 m³. Application was completed using a motorized or hand-pump sprayer or mister. Examples 79, 108, and 109 afforded greater than about 90% reduction of litter beetles and larvae, compared to the number of beetles and larvae before treatment.

Test materials and compositions disclosed herein were applied to the air and surfaces in chicken and swine production facilities that were infested with flying insects, including house flies (Musca domestica) and other types of flying insects, at a rate of about 1.5 gallons to about 3 gallons per 1,000 square feet. Examples 74 and 75 provided greater than about 90% reduction of adult flying insects when applied as a spray in infested production facilities.

### Example 3

Test compositions and formulations were applied to droppings from chickens and a dose rate of about 0.1 to about 2 weight percent relative to the weight of droppings. The droppings were infested with maggots, and the emergence of adult flies and mortality of emerged flies were measured relative to untreated control groups. Example 129 afforded greater than about 90% control of fly larvae (M. domestica) when applied to infested chicken droppings.

### Example 4

Test compositions and materials were applied to red mites (D. gallinae) in a container at a rate of one spray (sufficient to dampen the mites and surface) with a hand sprayer. Examples 72, 73, 78, 79, 112, 114, and 116 afforded greater than about 90% mortality of red mite.

Test compositions and materials were applied to chicken production facilities infested with red mites, at a rate of about 30 kg to about 60 kg per 1,000 m², using a backpack or handheld duster, sprayer, or mister. Examples 72, 73, 78, 79, and 116 afforded greater than about 80% control of red mites when applied to infested chicken production facilities.

### Example 5

In some embodiments, provided herein are several arthropod, insect and internal parasite control blends comprising a synergistic combination, and in various combinations. Such can be effective in controlling internal parasites (for example, helminths and protozoa) in humans and animals and in controlling parasites and pests in agricultural production. These blends can be encapsulated (8) to enhance efficacy.

In some embodiments, provided herein are blended formulations for use against internal parasites for animals and humans and other pests, including data presented hereinabove. In one embodiment, sporozoites of Eimeria spp. in culture were treated with test materials at a rate of 0.01 to 2 as a weight percent of the culture medium. Examples 119-124 afford >90% control of sporozoites of Eimeria spp.

Chickens were treated with test materials mixed into feed at a dose rate of 20-1000 mg test material per kilogram body weight per day. The treated chickens were inoculated with Eimeria spp. oocysts, and treatment with feed containing the test material was continued for 5-14 days. Examples 130-139 afford up to 25% improvement in feed conversion ratio of in chickens that are infected with Eimeria spp., when compared to infected, untreated chickens.

Eimeria spp. oocysts were isolated in culture and treated with test materials at a rate of 0.01 to 2% as a percentage of the culture medium. Example 79 affords >70% sporulation of mortality of the Eimeria spp. oocysts.

Roundworms of the genus Ascaris were treated in culture with test materials at a rate of 0.01 to 2% as a percentage of the culture medium. Examples 119-123 afford 100% mortality of Ascaris spp. roundworms in this testing.

### Example 6

Some embodiments the disclosed compositions and formulations can be effective at controlling, by killing or repelling, external parasites of humans and animals.

In one embodiment, cat fleas (C. fells) and sheep ticks (I. Ricinus) were treated with test materials by spraying at a rate sufficient to dampen the organisms and surface, Examples 76 and 29 afforded 100% control when sprayed on adult and immature stages of the cat flea (C fells) and castor bean tick (I. ricinus).

Repellency effect on the product was tested against cat fleas and sheep ticks. Mice were treated with test materials at a rate of 0.01-1 g per 50 g body weight by spraying onto the fur. The mice were placed into a test chamber and the pests released into a connected chamber where they could approach the treated mice. Examples 29 and 76 afforded >80% repellency of the cat flea (C. felis) and sheep tick (I. ricinus) compared to untreated controls.

Bed bugs were treated with test materials by spraying at a rate sufficient to dampen the organisms and surface. Examples 74 and 75 afforded 100% mortality when sprayed on bed bugs (C. lectularius).

### Example 7

Pests of stored gains were treated with test materials by spraying at a rate sufficient to dampen the organisms and surface. Example 79 affords >80% mortality of S. oryzae, R. dominica, and T. castaneum when applied as a spray.

### Example 8

Grape vines were treated with test materials by spraying plants infested with the insect green leafhopper at a rate sufficient to dampen the organisms and leaf surface. Examples 78 and 79 afforded 100% mortality of the green leafhopper.

Winged green aphids and Lixus weevils in petri dishes were treated with test materials by spraying at a rate sufficient to dampen the organisms. Example 77 afforded greater than 70% mortality of aphids and weevils after spraying.

Box tree plants infested with box tree moth were treated with test materials by spraying plants infested with the insect at a rate sufficient to dampen the organisms and leaf surface, Examples 83, 106, and 107 afforded greater than 90% mortality of box tree moth when sprayed on the leaves of box tree plants.

### V. REFERENCES

All references listed herein including but not limited to all patents, patent applications and publications thereof, scientific journal articles, and database entries are incorporated herein by reference in their entireties to the extent that they supplement, explain, provide a background for, or teach methodology, techniques, and/or compositions employed herein.

(1) How to Feed the World in 2050, FAO October 2009.

(2) Zhang, W., Global pesticide use: Profile, trend, cost/benefit and more; Proceedings of the international Academy of Ecology and Environmental Sciences, 2018, 8(1): 1-27

(3) Soil-transmitted Helminth Infections, World Health Organization, March 2, 2020.

(4) Nicolopoulou-Stamati et al., Chemical Pesticides and Human Health: The Urgent Need for a New Concept in Agriculture, Frontiers in Public Health 2016; 4: 148.

(5) Hawkins et al., The evolutionary origins of pesticide resistance, Biol Rev Camb Philos Soc. 2019 Feb; 94(1): 135-155.

(6) Isman and Machial, Chapter 2 Pesticides based on plant essential oils: from traditional practice to commercialization, Advances in Phytomedicine, Volume 3, 2006, Pages 29-44

(7) Plant Oils Fact Sheet, EPA, 1 Jul. 2001.

(8) Handbook of Encapsulation of Controlled Release, Munmaya Mishra, editor, CRC Press, 2016.

It will be understood that various details of the presently disclosed subject matter may be changed without departing from the scope of the presently disclosed subject matter. Furthermore, the foregoing description is for the purpose of illustration only, and not for the purpose of limitation.

1. A biocide or biopesticide composition having a repelling and/or killing effect against one or more pests or parasites comprising a blend of one or more essential oils and one or more carriers.

2. The biocide or biopesticide composition of claim 1, wherein the essential oils of the composition have a synergistic effect.

3. The biocide or biopesticide composition of claim 1, wherein the composition is effective in controlling internal parasites and/or helminths in humans and animals and helminths in crops.

4. (canceled)

5. The biocide or biopesticide composition of claim 1 wherein the blend comprises geraniol, clove oil, soap, isopropanol and water, optionally about 0.5% to about 3.0% geraniol, about 0.5% to about 3.0% clove oil, about 0.5% to about 3.0% soap, about 3.0% to about 8.0% isopropanol and about 80.0% to about 90.0% water.

6. The biocide or biopesticide composition of claim 1 comprising geraniol, soap, isopropanol and water, optionally about 0.5% to about 3.0% geraniol, about 0.5% to about 8.0% soap, about 3.0% to about 8.0% isopropanol and about 80.0% to about 95.0% water.

7. (canceled)

8. The biocide or biopesticide composition of claim 6, further comprising Thyme oil at about 0.5% to about 3.0%.

9. The biocide or biopesticide composition of claim 1 comprising geraniol, triethyl citrate and white mineral oil, optionally about 3.0% to about 8.0% geraniol, about 0.5% to about 3.0% triethyl citrate and about 90% to about 95% white mineral oil.

10. The biocide or biopesticide composition of claim 1 comprising geraniol, isopropyl myristate and white mineral oil, optionally about 3.0% to about 8.0% geraniol, about 0.5% to about 3.0% isopropyl myristate and about 90% to about 95% white mineral oil.

11. The biocide or biopesticide composition of claim 1 comprising geraniol, soap, ethyl lactate, glycerine, Isopropanol and mineral oil, optionally about 0.5% to about 8.0% geraniol, about 8.0% to about 14.0% soap, about 3.0% to about 8.0% ethyl lactate, about 0.5% to about 3.0% glycerine, about 0.5% to about 3.0% Isopropanol, and about 50% to about 80% mineral oil.

12. (canceled)

13. The biocide or biopesticide composition of claim 1, comprising geraniol, soap, Isopropanol and water, optionally about 0.01% to about 0.5% geraniol, about 0.5% to about 3.0% soap, about 3.0% to about 8.0% Isopropanol, and about 90% to about 95% water.

14. (canceled)

15. The biocide or biopesticide composition of claim 1, wherein the geraniol comprises geraniol having a purity ranging from about 5% to about 100%.

16. The biocide or biopesticide composition of claim 1, wherein the composition is a liquid, a solid, or in a substantially solid form.

17. The biocide or biopesticide composition of claim 1, comprising geraniol, Thyme oil, calcium silicate, calcium carbonate and sodium bicarbonate, optionally about 3.0 to about 8.0% geraniol, about 3.0% to about 8.0% Thyme oil, about 3.0% to about 14% calcium silicate, about 3.0% to about 40% calcium carbonate, and about 50% to about 90% sodium bicarbonate.

18. (canceled)

19. The biocide or biopesticide composition of claim 1, comprising one or both of thymol and sodium caseinate, and optionally one or more of linalool, carvacrol, para-cymene, and alpha-pinene.

20. The biocide or biopesticide composition of claim 19, comprising thymol at about 50% to about 100%.

21. The biocide or biopesticide composition of claim 19, comprising thymol at about 3% to about 50%, and sodium caseinate at about 80% to about 90%.

22. (canceled)

23. (canceled)

24. The method of claim 33, wherein the biocide or biopesticide composition is administered through a feed or foodstuff consumed by the subject, optionally a human or animal.

25. (canceled)

26. A method of treating an infection or infestation of invertebrate pests in a subject comprising administering the biocide or biopesticide composition of claim 1 to a subject, and optionally wherein the subject is a human or animal.

27-29. (canceled)

30. A method of treating an infestation of invertebrate pests in a domestic animal in need of treatment, comprising feeding the animal feed feed comprising the biocide or biopesticide composition of claim 1.

31. (canceled)

32. (canceled)

33. A method of repelling and/or killing on or more pests or parasites in a subject in need thereof, comprising administering to the subject a biocide or biopesticide composition comprising a blend of one or more essential oils and one or more carriers.
